(19) United States
(12) Patent Application Publication
(10) Pub. No.: US 2011/0082459 A1
(43) Pub. Date: Apr. 7, 2011

(54) STRUCTURED STERNAL INCISION

(76) Inventor: Dan Aravot, Haifa (IL)

(21) Appl. No.: 12/595,965

(22) PCT Filed: May 7, 2008
(86) PCT No.: PCT/IL2008/000632

§ 371 Date: Oct. 15, 2009

(51) Int. Cl. A61B 17/16

(52) U.S. Cl. 606/79

(57) ABSTRACT

A method of performing a sternal incision is disclosed. The method includes the employment of a surgical appliance for performing structured sternal incision. The surgical appliance is characterized by having an incising member for incising the sternal tissue and an actuating mechanism therefore. Upon actuating the incising member and driving the incising member of the surgical appliance relative to the sternum, the pattern of the structured sternal incision is formed.

### CROSS-REFERENCE TO RELATED APPLICATIONS

The present application claims the benefit of priority to U.S. Provisional Patent Application Ser. No. 60/916,591 filed 8 May 2005, entitled “STRUCTURED STERNUM INCISION”; the aforementioned application is incorporated herein by reference.

### FIELD OF THE INVENTION

The present invention relates to surgical appliances and methods in general and more particularly to a surgical appliance and method for performing structured sternal incision.

### BACKGROUND ART

The sternum is an elongated, flattened bone, forming the middle portion of the anterior wall of the thorax. Its upper end supports the clavicles, and its margins articulate with the cartilage of the first seven pairs of ribs. It consists of three parts, named from the neck downward, the manubrium, the body, and the xiphoid process. Its average length in an adult is approximately 17 cm, and is slightly less in a female adult. During surgery involving thoracic organs such as the heart, lungs, esophagus and aorta, it to may be required to split the sternum to provide sufficient access for the surgeon. A partial or median sternotomy is a procedure by which a saw or other cutting instrument is used to make a midline, longitudinal incision along a portion of, or the entire axial length of the patient's sternum; median sternotomy is used for most cardiac operations. When the entire sternum is cut in half longitudinally, its sternal halves are spread apart laterally, exposing the mediastinum structures. As a result, a large aperture is formed in the thoracic cavity, which permits optimal surgical access to the heart and the large blood vessels. Once the operation is completed, the two sternal halves are recombined by engaging the sternal halves in a face-to-face relationship, keeping them mutually compressed for as long as the sternum heals. Whereas traditionally, several means, such as Mersilene timbres, steel wires, metal and plastic bands, nitinol clamps, etc. are used to assure the sternal stability and recovery of the patient, the most typical current method of doing this is to use steel wires. The wire sutures are wrapped around the sternal halves by passing them through the intercostal spaces adjacent to the sternum. They may also be pierced through both halves of the sternum, particularly near the manubrium, to reduce axial motion of the sternal halves relative to each other. Numerous patents have been issued on various technologies all of which are aimed to provide solutions for rejoining and closing the sternal halves. U.S. Pat. No. 3,802,438 discloses sternal closure with wire sutures in conjunction with a splice plate. In U.S. Pat. No. 4,583,541, wire bands are used in concert with an elongated board, placed at the front of the sternum. Other representative examples are described in U.S. Pat. Nos. 4,201,215; 5,356,417; 5,462,542; and 6,007,538.

Despite its widespread use, medial sternotomy and subsequent sternal fixation is not without its morbidity and mortality. The complications usually arise due to sternum instability range from the sternal wound and prolonged thoracic pains, which cause inconveniences and related respiratory disorders, up to the sternal dehiscence (i.e. spontaneous bursting open of the sternum) occurring at about 2.4% incidence and mediastinitis at about 0.25% incidence, as disclosed in US patent application 2002 0165548. In addition, sternal malunion and nonunion contributing to excessive sternotomy site movement worsens postoperative pain leading to decreased inspiratory effort. An increasing number of patients with coexisting chronic obstructive pulmonary disease (COPD) or cough, diabetics, patients on steroids, older population with osteoporosis and moribund patients—recognised risk factors for impaired wound and bone healing—are now routinely undergoing surgery.

Fortification of sternum's stability and resultant reduction of the risk of sternal dehiscence and sternal infection following a sternotomy remains a challenge to the cardiac surgeon. As stems from the foregoing, several authors have investigated various devices and methods for sternal fixation following median sternotomy; however, as of yet neither method nor device or system facilitating an improved sternal fixation without employing various extrinsic accessories, inherently, due to structured geometry of the sternal incision was not suggested.

### SUMMARY OF THE INVENTION

There are provided in accordance with some embodiments of the present invention surgical appliances that are capable of performing a structured sternal incision and a method of using the same.

The object of the present invention is to teach a method of performing a structured incision of a sternum, with an increased interface contact area, having a predetermined pattern and/or depth. As the result of the structured incision the sternum is sectioned into two halves. The structured pattern of the edges resulting from the surgical method provides for a steadier mutual positioning of the two sternal halves and promotes accurate alignment of them. After the halves are aligned and set together, they are tightened together by means of stainless steel or nitinol wire or any other means known in the art and hence remain basically motionless one with respect to the other, promoting thus quicker healing of the sternum and better recuperation.

### BRIEF DESCRIPTION OF THE DRAWINGS

The present invention will be understood and appreciated more fully from the following detailed description taken in conjunction with the appended drawings in which:

FIG. 1 is a schematic top view diagram representing the sternum, the abscissa axis and the ordinate axis, in accordance with the present invention;

FIG. 2A is a schematic diagram representing an exemplary trajectory of the structured incision;

FIG. 2B is a schematic diagram representing another exemplary trajectory of the structured incision;

FIG. 2C is a schematic diagram representing yet another exemplary trajectory of the structured incision;

FIG. 2D is a schematic diagram representing yet another exemplary trajectory of the structured incision;

FIGS. 3A-B are isometric views of the stencil of some embodiments of the present invention;

FIG. 3C is a top view of the stencil of some embodiments of the present invention;

FIG. 3D is a side view of the stencil of some embodiments of the present invention;

FIG. 4A is a schematic side view cross-sectional diagram s representing an incising member prior to it incising the sternal tissue, in accordance with an exemplary embodiment of the incising member of the surgical appliance of the present invention;

FIG. 4B is a schematic side view cross-sectional diagram representing an incising member after it has incised the sternal tissue for to some time;

FIG. 5A is a schematic isometric view from below representing an incising member; in accordance with an exemplary embodiment of the incising member of the surgical appliance of the present invention;

FIG. 5B is a schematic isometric view from above representing an incising member;

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention.

### DISCLOSURE OF THE INVENTION

Illustrative embodiments of the invention are described below. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which vary from one implementation to another. Moreover, it will be appreciated that such a development effort might be complex and time-consuming, but would nevertheless be a routine undertaking for those of ordinary skill in the art having the benefit of this disclosure.

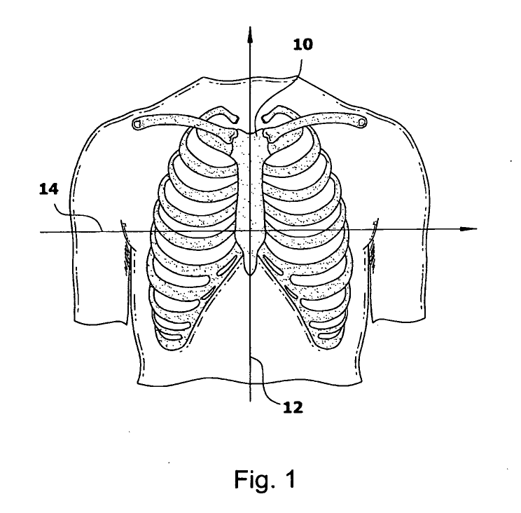

According to the present invention, the structured incision is a is surgical section implemented in the three dimensional space defined geometrically, for the sake of simplicity of describing, by three axes as follows: first the dimension along the sternum's length, henceforth abscissa axis; second, the dimension along the sternum's width, henceforth ordinate axis; and third, the depth of the incision inside the sternum along the third of the coordinate axes. Reference is now made to FIG. 1 wherein depicted is schematic representation of sternum 10, abscissa 12, and ordinate 14.

The Method of Performing the Structured Sternal Incision

According to the method of the present invention, the structured incision is created by an incising member (cutting means) that advances along a path to cut the sternum. The pattern of the path along which the incising member is to advance defines the shape of the structured incision. The pattern of the path can be either pre-planned or spontaneously generated in real time, for instance by the directing hand movement of a surgeon.

In one embodiment of the method according to which the pattern is spontaneously generated by the surgeon (hereafter spontaneously generated pattern or SGP) requests from the surgeon the skills of designing the optimal pattern for a patient off-the-cuff, concurrently with performing the incision. Although SGP may bear an increased risk of human error or mal-considering of all the relevant patient-specific medical characteristics, it may constitute a preferred embodiment of the method of the present invention for example in emergency procedures when it is urgent to establish an access into the thoracic cavity. Furthermore, SGP can be implemented with previously marked visual reference and/or preset stencil and/or redundantly combined with a supervision or control of a robotic surgical appliance, as will be elaborated infra.

According to another embodiment of the present invention, the pattern of the path is pre-set ahead of the operation (hereafter preset pattern or PS) and implemented by means of a stencil.

According to yet another embodiment of the present invention, the pattern of the path is pre-planned ahead of the operation (hereafter pre-planned pattern or PP). Implementing the PP is preferred usually in such cases as diagnosis is applicable and more thorough and methodical planning of the pattern is desired. It is further disclosed that the PP and/or PS, as referred herein, may include information regarding the position, shape and size of holes to be created in the sternum for tightening together the halves thereof, after the sternotomy, by a wire or any other means known in the art.

For that purpose, the sternum of a patient is initially examined and pertinent medical characteristics of the sternum are acquired. In a non-limiting manner, the sternum can be examined by any of the following means: visual and/or photographic inspection, computerised tomography (CT), magnetic resonance imaging (MRI), angiographic imaging, X-ray imaging, positron emission tomography (PET), single photon emission computerized tomography (SPECT), functional magnetic resonance imaging (fMRI), and ultrasonic imaging. Ultrasonic imaging, inter alia, can be performed by engaging ultrasonic probes to particular points on sternum's surface. Upon engaging an ultrasonic probe to a particular point, the spatial and/or Cartesian coordinates (Cartesian coordinates as referred herein include the coordinates of a point along the abscissa and ordinate axes) representing the location of a point on sternum's surface can be acquired, thereby providing a digitalized information regarding the surface topography of the sternum, simultaneously with acquiring digitalized information about the thickness of the sternum at this given point, provided by the ultrasonic probes that emit a mechanical wave into the sternum's tissue and collect its reverberation therefrom.

According to a particular condition of the patient, a complete three- or two-dimensional model of sternum's geometry can be acquired, for instance by subjecting the patient to a computerised tomography (CT) scan. The three- or two-dimensional evaluation of the sternum, performed with the purpose of acquiring a three- or two-dimensional sternal model (hereinafter referred to as acquired three- or two-dimensional sternal model or ASM), can be justified particularly if a somewhat complicated medical condition is envisaged and decided to be employed upon preliminary examination.

Alternatively, the three- or two-dimensional sternal model can be to generated by a computer. Accordingly, the computer is pre-programmed with a flexible model representing a typical three- or two-dimensional geometry of human sternum. The usage of such pre-programmed flexible models is known in the art and exemplarily represented in EP1100377 entitled “SYSTEM FOR DYNAMICALLY CORRECTING A THREE-DIMENSIONAL GRAPHICAL MODEL OF A BODY REGION”, the content of which is incorporated herein by this reference. Upon providing some characteristics of the sternum of an individual patient, the flexible model is scaled and/or adjusted to generate a personalized three- or two-dimensional model of the sternum (hereinafter referred to as generated three- or two-dimensional sternal model or GSM) that corresponds patient's individual characteristics. The GSM can be justified if a routine sternotomy procedure is expected and decided to be employed upon preliminary examination.

Exemplarily, the characteristics of the sternum of an individual patient, inter alia, include relative distances between particular points of the sternum that can be located thereon and/or identified via a suitable imaging modality. After determining of the relative distances between these particular points, the flexible model is scaled and/or adjusted so that the respective distances in the flexible model approximately assume the individual values compliant with patient's individual characteristics.

Exemplarily, the flexible model has predefined coordinate points, which correspond to particular points on the articulated surface of the sternum that can be located thereon and/or identified via a suitable imaging modality. Upon determining of the spatial and/or Cartesian coordinates of to these particular corresponding points on patient's sternum, the flexible model is scaled and/or adjusted so that the respective predefined coordinate points thereof are urged to approximately coincide with each other; thereby the model assumes geometry compliant with patient's individual characteristics.

Spatial and/or Cartesian coordinates of a particular point on the articulated surface of the sternum and/or the relative distances between particular points can be determined by the surgeon, for instance by engaging an ultrasonic or some other probe thereto.

If an ultrasonic probe is used for determining the spatial and/or Cartesian coordinates of a particular point on the articulated surface of the sternum and/or the relative distances between particular points, the complimentary information provided by the probes regarding the thickness of the sternum can be utilized for a more accurate and sophisticated scaling and/or adjusting of the flexible model.

Preferably, the values of the spatial and/or Cartesian coordinates of particular points on patient's sternum and/or relative distances between particular points thereof and/or some other values provided for scaling and/or adjusting the flexible model, has predefined deviation tolerance that serves two distinct purposes. Firstly, upon obtaining the individual values of the patient, they are compared to the predefined deviation tolerance and the probability for creating a sufficiently correct model is thereby calculated; thus if patient's individual values deviate from the predefined tolerance the relative probability for creating a sufficiently correct model may not be adequate. Secondly, the aforementioned values comparing and probability to calculating can be used as independent diagnostic measure devising whether the GSM or ASM should be employed.

Upon scaling and/or adjusting the flexible model, by whichever means, the GSM is consolidated and further used for planning the PP.

After the pertinent medical characteristics were obtained and/or is a three- or two-dimensional ASM or GSM was acquired or generated, various parameters of the PP, such as the geometry and position thereof relatively to the sternum, are premeditated in the aforesaid ASM or GSM, by a practitioner and/or devised by medicinal computer software implemented for such task, taking into account various patient-specific characteristics as will be described below. The PP than can be visually marked on the surface of the sternum (hereafter visually marked PP or VMPP). The ASM or GSM is subsequently updated and the PP is reproduced therein. The PP as referred herein in a non-limiting manner can be defined by the following parameters:

1—the coordinates of the incision and/or the coordinates of the holes on the plane defined by the abscissa and ordinate axes, namely the pattern of the PP;

2—the coordinates of the incision and/or the coordinates of the holes along the third coordinate axes;

3—the angular orientation of the incising member, between the third coordinate axis and the abscissa-ordinate plane at a given point of the incision and/or a hole, namely the angular orientation of the PP;

4—the length of the incision to de performed into the thickness of the sternum, along the third coordinate axis or along the axis representing the angular orientation of the PP, at a given point of the incision and/or a hole, namely the depth of the incision;

5—the width of the incision and/or the diameter of a hole;

6—the deviation of the parameters 1 to 5 tolerated for a given sternotomy procedure, namely the tolerance for the aforementioned parameters.

The updated solid model containing the parameters of the PP is then superimposed with the sternum of the patient. The model may have for instance reference points that correspond to particular points on the articulated surface of the sternum and can be allocated thereon. A point on the articulated surface of the sternum can be allocated by the surgeon, for instance by engaging an ultrasonic or some other probe thereto. After that a plurality of static points were allocated on the articulated surface of the sternum, the corresponding reference points of the solid model are superimposed therewith. If the GSM is used, determining the spatial and/or Cartesian coordinates of a particular point on the articulated surface of the sternum and/or the relative distances between particular points, for scaling and adjusting the flexible model, can be performed concurrently with allocation of the static reference points, for superimposing a sternal model containing the parameters of the PP. Thus, upon engaging a probe to a predefined point on the sternum, the coordinates of the point can be recorded and then used as a spatial and/or Cartesian reference for the surgical appliance of the invention, i.e. allocation of a reference point; these coordinates relatively compared to the coordinates of other points, however, can also be used as an input for the algorithm that scales and adjusts the flexible model.

Preferably, the graphic representation of the PP is generated and visually presented on the sternum's surface, for instance by a laser beam projected thereon by the surgical appliance of the present invention, and/or on a graphical user interface of the aforementioned appliance. The position and geometry of the PP can then be evaluated the medical personnel. If the position and geometry of the PP, as presented, comply with the professional opinion of the medial personnel, the PP can be affirmatively accepted and either visually marked on the surface of the sternum, namely VMPP approach, and/or stored in a digital memory medium (hereafter digitally stored PP or DSPP approach). Preferably, the visual marking of the PP on the surface of the sternum is performed by means of the aforementioned laser beam projected thereon by the surgical appliance but this time with an augmented intensity; thereby scorching on the sternum's surface a visual representation of the PP. Alternatively or additionally a dye marking can be employed for VMPP.

VMPP and/or DSPP are used as a reference for the positioning and leading of the incising member of the surgical appliance of the present invention according to the PP parameters detailed supra and to thence tracing the PP with the incising member while performing the structured incision.

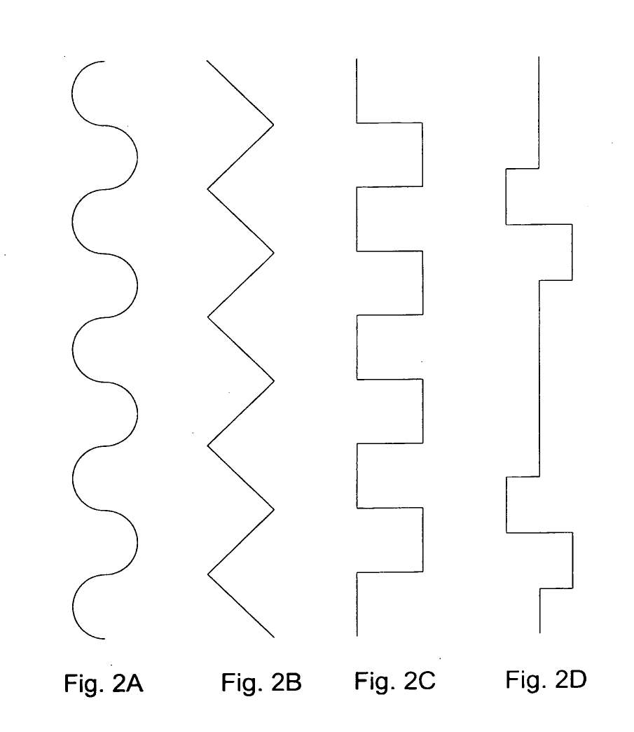

According to the present invention, the structured incision can assume a variety of patterns, shapes or profiles. Noticeably, the pattern of the structured incision, contradistinctively to the methods known in the art, is not linear or completely straight. The pattern of the structured incision thus can assume in a non-limiting manner sinusoidal, toothed, jagged, serrated, notched, crenelated or zigzagged shapes. Reference is now made to FIGS. 2A-2D showing several exemplary two-dimensional patterns of the is structured incision on the abscissa-ordinate plane, according to the method of the present invention. Parameters that are taken into consideration in the design pattern the of the structured incision in a non-limiting manner include: the margin from the incision edge to the edges of the sternum on the abscissa-ordinate plane; the thickness map of the sternum; the preferred geometry of the pattern that will suit the individual medical case, etc.

According to some preferred embodiments of the method of the present invention, the pattern of the structured incision is characterized in three dimensions rather than in the two dimensions of the abscissa and the ordinate axes. Such three-dimensional pattern of the structured incision (hereafter 3DP) can be achieved by changing the orientation of the incising member, tilted sideways and/or penetration level of the incising member into the sternum. 3DP provides for preventing the mutual movement of the two reunited halves of the sternum, as may be expected in the case of two-dimensional pattern, but also along the third coordinate axis, which shall encourage quicker healing of the sternum and improved recuperation.

According to some preferred embodiments of the method of the present invention, the holes created in the sternum for joining and tightening of the halves after the sternotomy are to be created before the sternotomy procedure.

According to some preferred embodiments of the present invention, the incising member is oriented to incise from the inside of the thoracic cavity outwards the patient's body; thus minimising the risk of an iatrogenic injury that may be caused by the incising member unintentionally incising or damaging tissues.

### The Surgical Appliance for Performing the Structured Sternal Incision

### Embodiment No 1

In accordance with some embodiments, the surgical appliance of the present invention, in its overall shape, may somewhat resemble a typical electrically-powered hand saw, otherwise known as Jigsaw, but instead of operating a saw-blade, the surgical appliance of the present invention operates an incising member, which is characterised by the ability to incise through the sternum laterally, while oriented substantially perpendicularly to the abscissa and the ordinate axes as defined hereinabove. The surgical appliance in this case typically consists of an incising member, actuating mechanism for operating the incising member, and firm housing having a gripping handle encompassing aforesaid components. The driving of the incising member of the surgical appliance relatively to the sternum is achieved by the surgeon manually manipulating the incising member by means of muscle force. This embodiment can be beneficial in implementing SGP as neither prior diagnosis nor methodical planning of the pattern of the structured incision is involved with SGP. If PP is implemented with this embodiment, the VMPP is beneficially applicable since it does not require the allocation of static reference points typically associated with DSPP as will be described below. However, according to some embodiments of the method VMPP can be combined with DSPP, in such manner that DSPP serves as a validating reference for the actions of the surgeon while VMPP is employed as the primary reference.

### Embodiment No 2

In accordance with some embodiments, the surgical appliance of the present invention includes a stencil that assists in positioning and orienting the incising member according to a preset pattern PS. Examples of the incising member to be used with the stencil in a non-limiting manner include: the incising member as of the embodiment No 1 of the surgical appliance detailed supra; a typical drill as known in the art for drilling the holes in the sternum used for tightening together the halves thereof after the sternotomy; a typical sternotomy blade as known in the art for incising the sternum into two halves.

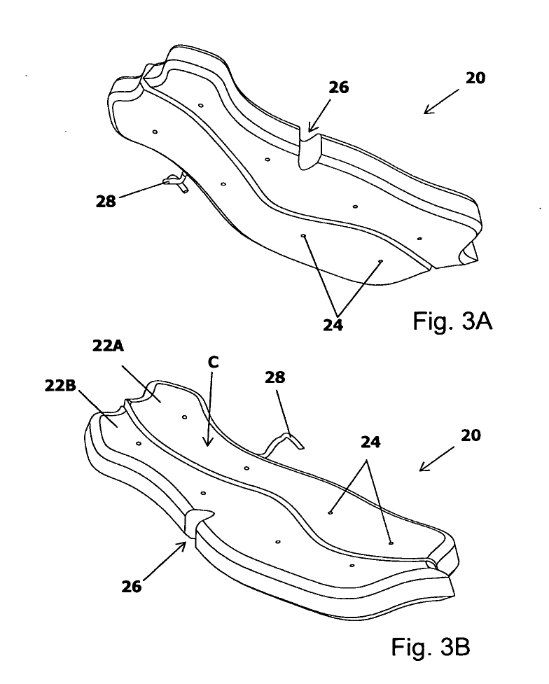

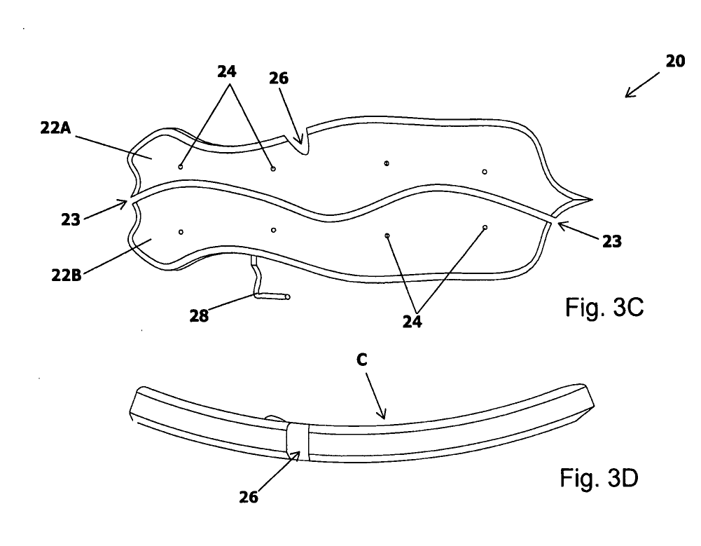

Reference is now made to FIGS. 3A-D, showing exemplary stencil 20 in accordance with some embodiments of the present invention. Stencil 20 includes two subunits 22A and 22B defining pathway 23 therebetween having a PS pattern. Stencil 20 has somewhat convex inner face C for adjoining to the upper face of the sternum. It should be noted to however that any shape of inner face C that conforms with the sternum's structure can be employed. Pathway 23 is used for positioning and orienting incising member (not shown) according to the PS pattern of stencil 20.

Stencil 20 includes apertures 24 for positioning and orienting a drill (not shown) used for drilling the holes in the sternum, subsequently used for tightening together the sternum halves, after the sternotomy.

Stencil 20 further has groove 26 and articulated arm 28 for affixing stencil 20 to the sternum (not shown). It should be noted however that any structural elements and/or features, integral or modular can be used with the stencil of the present invention.

Preferably, the practitioner is to be provided with a collection of preset stencils that are designed to comply with a specific type of patients, such as kids, teenagers, women, patients exceeding a predetermined height, etc. Upon the decision of the practitioner particular stencils is selected for use.

It is further disclosed that certain stencils can be adopted to be used with the specific incision means typically used in a typical sternotomy operation, known in the art for incising the sternum into two halves. The PS pattern is accordingly planned in such a manner that the surgeon will be able to guide a typical sternotomy blade and/or blade of a typical sternal saw by a directed hand movement implementing the PS pattern of the stencil.

### Embodiment No 3

This embodiment of the surgical appliance is a robotic implement consisting of a platform, a plurality of gripping-fixation arms and at least one incising member; conjointly with controlling and operating mechanisms therefore, mounted on the aforesaid platform. The movement of the gripping-fixation arms is characterized by several, controllable degrees of freedom. The gripping-fixation arms extend from the platform and adjustably fix or fasten to the sternum, the ribs or elsewhere to a body of a patient intended to undergo an operation. After the gripping-fixation arms have been set against the tissue or organ they are fastened thereto and may be further immobilized by precluding some or all the possible degrees of freedom. Consequently, the platform becomes substantially immovable relatively to the sternum; hence the surgical appliance and the body of the patient share the same coordinate system. This embodiment of the surgical appliance is particularly beneficial for implementing the PP approach. If a VMPP is employed the PP is marked on the sternum's surface.

For implementing the DSPP approach, on the plane defined by the abscissa and the ordinate axes and/or on the sternum's surface a plurality of static reference points is allocated.

After having pre-planned the preferred parameters of the PP, by whichever means, the superposition and orientation of the PP is predetermined on the aforesaid abscissa-ordinate plane and/or along the third dimensional axis with coordinates made to the aforesaid plurality of static reference points. The superposition of the PP and its coordinates on the abscissa-ordinate plane and/or along the third dimensional axis, thereafter, stored as DSPP in any type of a digital memory medium such as RAM memory medium, ROM memory medium, flash memory medium, magnetic medium or an optic storage device.

In accordance with the DSPP approach, guiding of the incising member is carried out by employing the coordinates along the abscissa and ordinate axes and/or along the third dimensional axis, thus controlling of the orientation and position of the incising member. A structured incision is achieved by the controlling and operating mechanisms of the gripping-fixation arms and/or the incising member/s of the surgical appliance. The controlling and operating mechanisms position and/or orientate the incising member/s of the surgical appliance to comply with parameters of the DSPP stored in the memory medium and trace the graphic-representation of the DSPP with the incising member thus performing the structured incision.

In accordance with the VMPP approach, the surgical appliance further consists of an optical sensing means that is able to observe the mark and communicate with the aforesaid controlling and operating mechanisms of the gripping-fixation arms and/or the incising member/s of the surgical appliance providing them with reference for the correct position of the incising member on the abscissa-ordinate plane and/or sternum's surface.

### Embodiment No 4

According to yet some other embodiments, the surgical appliance of the present invention is a stationary robotic implement situated in a surgery room and typically but not necessarily mounted on the floor, the wall or elsewhere in the room, or on a special supporting structure surrounding a surgery bed. Preferably the robotic implement is mounted on a base-platform that is movable among rooms and affixed to the floor by lifting itself up therefrom by means of extendable supporting legs frictionally engaging the floor. The robotic implement includes a structural framework having operative module, and at least one incising member, conjointly with controlling and operating mechanisms therefor mounted on the aforesaid framework. According to these embodiments, the movement of the operative module of the structural framework is characterized by several controllable degrees of freedom, so that the aforesaid controlling and operating mechanisms can confer to the framework a certain three dimensional structure in a given time. This provides for positioning the incising member and guiding it as required. Noticeably, the structural framework remains mobile relative to the sternum during the operation and hence the surgical appliance and the body of the patient do not share the same coordinate system.

In accordance with the DSPP approach, preferably, initially, a plurality of static reference points is allocated on the plane defined by the abscissa and the ordinate axes and/or on the sternum's surface.

After having pre-planned the preferred parameters of the PP, by whichever means, the superposition and orientation of the PP is predetermined on the aforesaid abscissa-ordinate plane and/or along the third dimensional axis with coordinates made to the aforesaid plurality of static reference points. The superposition of the PP and its coordinates on the abscissa-ordinate plane and/or along the third dimensional axis, thereafter, stored as DSPP in any type of a digital memory medium such as RAM memory medium, ROM memory medium, flash memory medium, magnetic medium or an optic storage device.

In accordance with the DSPP embodiments, controlling the coordinates of the incising member along the abscissa and ordinate axes and/or along the third dimensional axis, controlling of the orientation of the incising member, and driving the incising member of the surgical appliance according to the coordinates of the superposed DSPP, which in fact is tracing the DSPP with the incising member thereby performing the structured incision are achieved by the controlling and operating mechanisms of the structural framework and/or the incising member/s of the surgical appliance. The controlling and operating mechanisms position and/or orientate the incising member/s of the surgical appliance in accord with parameters of the DSPP stored in the memory medium and trace the graphic-representation of the DSPP with the incising member thus performing the structured incision.

In accordance with some embodiments of the VMPP method, the surgical appliance further includes an optical sensing means that is able to observe the mark and communicate with the aforementioned controlling and operating mechanisms of the structural framework and/or the incising member/s of the surgical appliance providing them with reference for the correct positioning of the incising member.

Since the structural framework of the surgical appliance remains mobile relative to the sternum during the operation, the position of aforementioned incising member/s relative to the aforementioned plurality of static reference points is constantly and repeatedly verified and validated by the aforementioned controlling and operating mechanisms. If the sternum of the patient has moved, the controlling and operating mechanisms of the robotic implement update and adjust the conformation of the operative module of the structural framework and position the incising member to comply with a given coordinate and/or orientation in accord with the DSPP and/or VMPP.

The current embodiment of the surgical appliance can be beneficially applied in implementing the 3DP since the operative module of the structural framework provides for the flexible positioning and orienting the incising member relatively to the sternum.

### Embodiment No 5

In accordance with some preferred embodiments, the surgical appliance of the present invention is characterized by having additional auxiliary functionalities for performing a complete sternotomy procedure. Thus several changeable tools can be operationally connected and actuated by the operative module, such as the module of the structural framework of the third embodiment detailed supra.

Firstly, an ultrasonic probe can be operationally connected to the operative module and engaged to the sternum, thereby providing the spatial and/or Cartesian coordinates of the point that can be used as a reference for the surgical appliance of the invention, i. e. allocation of a reference point; and/or providing an input for the algorithm that scales and adjusts the flexible model.

Secondly, a marking means can be operationally connected to the operative module for marking of VMPP.

Thirdly, a boring tool can be operationally connected to operative module for drilling the holes in the sternum for tightening together the halves thereof after the sternotomy.

Fourthly, a thermal ablating tool can be operationally connected to the operative module for thermally ablating the periosteum or elsewhere alongside the interior surface of the holes and/or the surface of the sternum cut after the incision; thereby preventing the bleeding of the ablated sternal tissue therealong.

Fifthly, an incising member can be operationally connected to the operative module for performing structured incision.

Sixthly, a wax-depositing tool can be operationally connected to the operative module for pressing bone wax into the bone marrow, the periostea or elsewhere alongside the interior surface of the holes and/or the surface of the sternum cut after the incision, as a hemostatic agent to reduce the bleeding therefrom.

The surgical appliance preferably includes a graphical user interface for visually presenting the graphic representation of the PP on sternum's surface. The surgical appliance preferably includes a laser beam projecting means for visually presenting the graphic representation of the PP on sternum's surface and/or scorching the sternum's surface a thereby marking the visual representation of the PP.

The surgical appliance may further include one or more vibro-acoustic sensors, such as microphones or ultrasonic probes, for monitoring the mechanical waves generated upon the interaction of the incising member with the sternal tissue. Such mechanical waves can be generated upon interaction with a mechanical incising member/s, such as the incising member detailed in examples number 1 and 4 to 7 infra, generated by a thermo-mechanical ablation induced by a laser beam detailed in example number 2 infra, or by a mechanical ablation induced by a jet of pressurized liquid detailed in example number 3 infra. The frequencies, intestines or other characteristic mechanical waves The alterations in this consumption that exceeded a predetermined threshold/s can be interpreted as indicative of an imminent proximity towards the end of the sternal tissue and the operation of the incising member can be accordingly adjusted, for instance by decreasing the intensity of incising member's action, and/or magnitude of the force applied to the incising member directed substantially transversely to the abscissa-ordinate plane, i.e. into the sternal tissue.

### Embodiment No 6

In accordance with some embodiments, the surgical appliance of the present invention is characterized by being operateable in several combined manual and automatic operational modes, as detailed infra. Thus the surgical appliance can include a gripping handle for the surgeon to exert his/her muscular force thereby positioning, orienting and/or actuating the incising member, as in Embodiment No. 1 supra, as well as and in conjunction with controlling and operating mechanisms for positioning, orienting and/or actuating the incising member, mounted on aforementioned structural framework and/or the operative module thereof, as in embodiment 4 supra, or on the aforementioned platform, as in embodiment 3 supra.

Additionally or alternatively the surgical appliance can include a hand-held direction controlling stick, otherwise known at the colloquial language as joystick, for the surgeon to apply his/her muscular force thereon, thereby controlling and operating mechanisms position, orientate and/or actuate the incising member according to the surgeon's will.

### Operational Modes of the Surgical Appliance

Preferably, the surgical appliance is operateable in several discrete or combined operational modes.

In automatic operational mode the position, orientation, actuation and/or the intensity of actuation of the incising member are controlled by the surgical appliance. Upon implementing the PP method the surgical appliance positions, orientates and actuates the incising member according to the predefined parameters of the PP thereby performing it.

In manual operational mode the position, orientation, actuation and/or the intensity of actuation of the incising member are controlled by the surgeon.

In automatically supervised manual operational mode the position, orientation, actuation and/or the intensity of actuation of the incising member are performed by the surgeon and concurrently monitored by the surgical appliance, in such manner that the surgeon is free to position, orientate, and actuate the incising member by his/her muscular force but the surgical appliance alerts the surgeon in advance of and/or with an occurrence of a particular event during the procedure. The occurrence of a particular is event can be for instance the deviation from a predetermined parameter of the PP exceeding the aforementioned tolerance thereof.

In automatically controlled manual operational mode the surgeon positions, orients, and actuates the incising member by his/her muscular force but the surgical appliance inter alias disables the action of the incising member, obviates some or all degrees of freedom of the incising member's movement and/or orientation of the incising member, limits the movement and/or orientation of the incising member, and immobilizes the incising member in advance of and/or with an occurrence of a particular event during the sternotomy procedure. The occurrence of a particular event can be for instance the deviation from a predetermined parameter of the PP exceeding the aforementioned tolerance thereof.

Thus particular instances of performing structured sternal incision may involve the concurrently implemented SGP, VMPP and DSPP methods. Thus if the PP was marked on the sternum (VMPP) but the surgeon spontaneously opts to use some alternative pattern (SGP) and the surgical appliance alerts or the surgeon according to the digitally stored references (DSPP), the three methods are implemented concurrently.

### Incising Members of the Surgical Appliance

A variety of different incising members can be employed with various embodiments of the present invention. The examples provided below serve to illustrate a few possible and some preferred means of incising through bone, all of which but not exclusively are in accordance with the present invention. A feature common to the incising members is that they are characterised by their ability to incise through bone, in general, and through a sternum particularly, laterally, while substantially oriented perpendicularly to the abscissa and the ordinate axes.

### EXAMPLE NO 1

One example of an incising member is a special driller/blade. The driller/blade is characterised by having an edged or pointed cylindrical surface; and by revolving around itself at a predetermined rotary speed it cuts/erodes the full thickness of the sternum, while being substantially oriented perpendicularly to the abscissa and the ordinate axes. Although the particular embodiments wherein the driller/blade is rotated are preferred, the driller/blade may also be operated by back and forth movements. In such case the driller/blade is still distinguished from saw-blades that are known in the art by having presumably a cylindrical shape and by its ability to incise through the sternum laterally, while substantially oriented perpendicularly to the abscissa and the ordinate axes. Obviously, the rotational movement can be optionally combined with back and forth movement, as for instance in several combined and or discrete operational modes; all to achieve the surgeons' specific goals. Optionally, according to this example, the surgical appliance may additionally be furnished with a driller/blade guard for preventing a driller/blade from unintentionally incising or damaging tissues.

According to this example the depth of the incision inwards the thickness of the sternum, along the third coordinate axis, is predetermined and customizably set prior to the operation.

### EXAMPLE NO 2

Another example of an incising member is a beam of coherent electromagnetic radiation in the ultraviolet, visible, infrared or any other regions of the spectrum, otherwise also known as a laser beam. Laser beams are commonly applied in many industrial and medical applications wherein they are used for cutting and incising through various materials and hence constitute some preferable choices for the incising member of the surgical appliance of the present invention. An example among numerous patents on lasers and laser related medical technologies can be found in U.S. Pat. No. 7,167,622.

According to this example the laser beam is directed onto the sternal tissue according to the PP of the structured incision and burns through the sternum with predetermined and/or regulated intensity. Additional optical sensing means may be employed in order to control the operation of the beam in order to prevent the beam from incising, burning or damaging thoracic tissues underneath the sternum.

### EXAMPLE NO 3

Another example of an incising member is a coherent jet created by stream of pressurised liquid, as particularly disclosed in U.S. Pat. No. 6,960,182. According to this example, a constellation wherein two or more converging jets are employed constitute some preferred embodiments of the incising member since in such constellation at the point of convergence the mincing force of the jets is distracted by each other which provides for incising the tissue to a predetermined depth, as disclosed in International Publication WO 2007 013076 the contents of which are hereby incorporated by reference herein.

### EXAMPLE NO 4

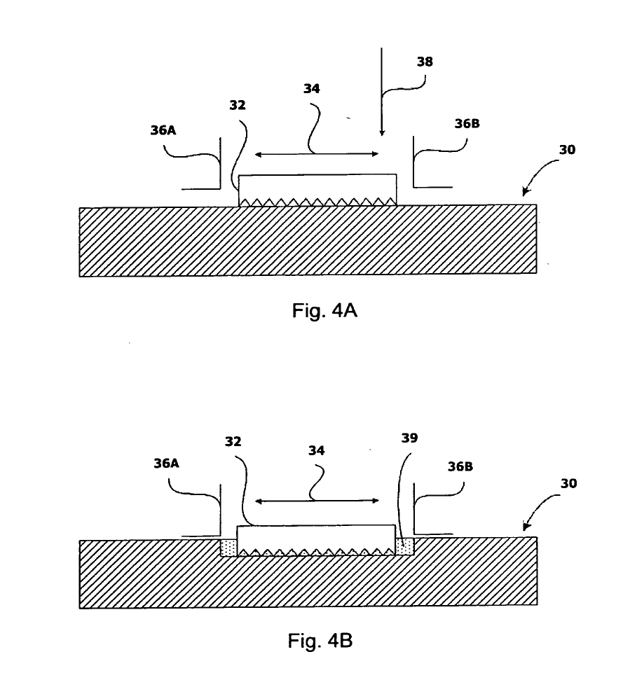

Another example of an incising member is one or more fret-saws. Reference is now made to FIGS. 4A and 4B wherein cross-sectional view of sternal tissue 30 and single fret-saw member 32 are presented. Fret-saw member 32 is repeatedly driven back and forth by a driving means (not shown) in the direction of arrow 34 while movement-restraining means 36A and 36B confine the movement of the fret-saw member 32 to the interval span between them. Conjointly, fret-saw member 32 is forced into sternal tissue 30 by a forcing means (not shown) in the direction of arrow 38. After having completed numerous movement cycles, fret-saw member 32 creates discrete rectangular incision 39 characterized by straight edges and having width substantially similar to the thickness of fret-saw member 32.

Alternatively or additionally, while being forced into the sternal tissue, the fret-saw or some other fret-saw like functioning member can be subjected to vibro-acoustic oscillations, preferably of an ultrasonic frequency, thereby eroding the sternal tissue underneath and creating straight-linear segment of the structured incision.

Reference is now made again to FIGS. 2B-2D; that each straight-linear segment of the exemplary structured incisions illustrated thereon can be created according to the foregoing description. Accordingly, a single fret-saw member can be repeatedly employed for successive creation of each individual straight-linear segment of the structured incision or an array of fret-saw members, which were previously aligned, can be operated concurrently for creation of all or several of the straight-linear segments of the structured incision at once.

### EXAMPLE NO 5

Another example of an incising member is a flexible continuous band-saw blade. Such a band-saw blade can be employed in order to create structured incisions without any sharp angles or turns such as the exemplary sinusoidally-shaped structured incision illustrated in FIG. 2A to which reference is again made. A plurality of cylindrical drum-members, arranged in a predetermined pattern, whereon and around which the flexible band saw blade is involuted, in order to confer to the flexible band saw blade the desired shape, constitutes a complementary component of the incising member, according to this example.

### EXAMPLE NO 6

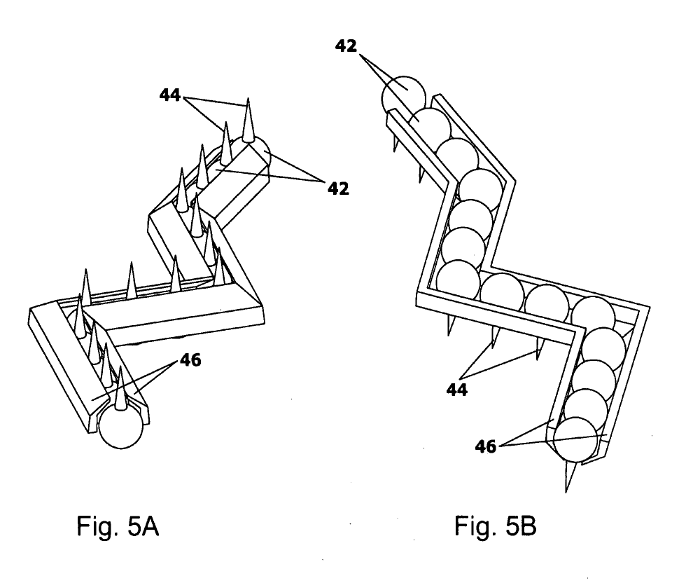

Another example of an incising member is an element which bears a functional resemblance to a chainsaw chain. However, contradistinctively to the conventional chainsaw chains that are pliable solely on a two dimensional plane, the incising member, according to the present example, is characterized by pliability in a three dimensional space. Reference is now made to FIGS. 5A and 5B wherein one possible variation of an incising member, in accordance with the current example, is illustrated. Spherical elements 42 have pointed conical tip 44 protruding out from structured rail 46, whereon spherical elements 42 are urged to slide by an urging means (not shown). Pointed conical tips 44 of spherical elements 42 are directed towards and pressed against the sternal tissue by the surgical appliance of the present invention; thus constantly and repeatedly scratching the sternal tissue, whereby structured incision having a shape substantially consistent with the path of rail 46 is created. Rail 46 can assume a variety of shapes and profiles, all accordingly to the desired shape to be conferred to the structured incision.

Other alternatives of an incising member according to the present example is a plurality of elements having pointed or sharp edge or tip are threaded onto a string and urged to slide on it or an incising member according to the present example as a plurality of elements, having pointed or sharp edge or tip, fastened one to the other thus constitutes a continuum characterized by sufficient flexibility and firmness.

According to some possible variations of the current example a sub-element with pointed or sharp edge or tip can be pulled out from or erected from the surface of the main element, such as spherical element 42, by a mechanical mechanism appropriate for such task.

### EXAMPLE NO 7

A typical drill as known in the art can be combined or used as additional incising member for drilling the holes in the sternum used for tightening together the halves thereof, after the sternotomy.

If a mechanical incising member/s is used, such as the incising member detailed in examples Nos. 1 and 4 to 7 supra, the power consumption of the mechanism actuating the incising member can be monitored. The alterations in this consumption that exceeded a predetermined threshold/s can be interpreted as indicative of an imminent proximity towards the end of the sternal tissue and the operation of the incising member can be accordingly adjusted, for instance by decreasing the magnitude the magnitude of the force applied to the incising member directed substantially transversely to the abscissa-ordinate plane, i.e. into the sternal tissue.

Noticeably, due to the obviation of the relative axial translation of the two reunited halves of the sternum along the ordinate axis inherently achieved by structured sternal incision, the amount the holes drilled in the sternum for tightening together the halves thereof after the sternotomy can be reduced to about four respective pairs of holes, contradistinctively to the about six to eight respective pairs of holes presently used in a standard sternotomy procedure.

It should be acknowledged that the present invention is equally applicable for cranial orthopaedic incisions as well as for any orthopaedic incision wherein obviation or reduction of the relative axial translation of the two reunited halves of the incised bone inherently achieved by structured incision thereof is desired.

It will be appreciated that the present invention is not limited by what has been particularly described and shown hereinabove and that numerous modifications, all of which fall within the scope of the present invention, exist. Rather the scope of the invention is defined by the claims which follow:

1. A method of performing a sternal incision comprising the steps of:
exposing the sternum of a patient;
providing a surgical appliance for performing structured sternal incision, said appliance comprising an incising member for incising the sternal tissue and an actuating mechanism therefor;
actuating said incising member, and
driving said incising member of said surgical appliance relative to said sternum forming a pattern in order to create a structured sternal incision.

2. A method of performing structured sternal incision as in claim 1, wherein said pattern has a shape selected from the group consisting of sinusoidal, toothed, jagged, serrated, notched, crenelated, zigzagged, or combination and variation thereof.

3. A method of performing structured sternal incision as in claim 1, further comprising the steps of:
examining the characteristics of the sternum of said patient and acquiring pertinent characteristics of the sternum;
pre-planning the desired pattern for the structured incision accordingly to said pertinent characteristics acquired at said step of examining.

4. A method of performing structured sternal incision as in claim 3, wherein said step of examining the characteristics of the sternum is to be selected from the group consisting of: visual inspection, computerized tomography (CT), magnetic resonance imaging (MRI), angiographic imaging, X-ray imaging, positron emission tomography (PET), single photon emission computerized tomography (SPECT), functional magnetic resonance imaging (fMRI), and ultrasonic imaging.

5. A method of performing structured sternal incision as in claim 3, wherein said step of pre-planning said desired pattern is devised by medicine software implemented for such task.

6. A method of performing structured sternal incision as in claim 3, further comprising a step of marking a graphic-representation of said pattern to be visualized on the surface of the sternum.

7. A method of performing structured sternal incision as in claim 3, further comprising a step of generating a digitized graphic-representation of said pattern and storing said digitized graphic-representation in a digital memory storage medium.

8. A method of performing structured sternal incision as in claim 7, wherein said memory storage medium is selected from the group consisting of RAM memory medium, ROM memory medium, Flash memory medium, magnetic medium, an optic storage device and combination thereof.

9. A method of performing structured sternal incision as in claim 7, further comprising the steps of:
defining a plane tangent to the upper face of the sternum by abscissa and ordinate axes;
allocating at least two static reference points on said plane;
predetermining the position of said graphic-representation of said pattern of said plane with coordinates being made to said at least two static reference points;
storing said graphic-representation of said pattern with its said coordinate on said plane in a memory storage medium;
positioning said incising member of said surgical appliance according to said graphic-representation of said pattern, and
tracing said graphic-representation of said pattern with said incising member.

10. A surgical appliance for performing structured sternal incision comprising:
an incising member for incising the sternal tissue;
an actuating mechanism for operating said incising member.

11. A surgical appliance as in claim 10, wherein said appliance further comprising a firm housing, having a griping handle.

12. A surgical appliance as in claim 10, wherein said incising member is selected from the group consisting of: a driller/blade, a beam of coherent electromagnetic radiation, a coherent jet created by stream of pressurised liquid at least one fret-saw member, a flexible continuous band-saw blade, a chainsaw chain-like element, a plurality of elements threaded onto a string, a plurality of elements fastened one to the other constitute a continuum characterized by sufficient flexibility and firmness, combination and variation thereof.

13. A method of performing structured sternal incision as in claim 1, wherein said surgical appliance further comprising a firm housing having a griping handle.

14. A surgical appliance as in claim 10, wherein said appliance further comprising:
a plurality of gripping-fixation arms, the movement of which is characterized by several, controllable degrees of freedom, and an actuating mechanism therefor, and
a platform whereon aforesaid components are mounted;
wherein said incising member is selected from the group consisting of: driller/blade, a beam of coherent electromagnetic radiation, a coherent jet created by stream of pressurised liquid, at least one fret-saw member, a flexible continuous band-saw blade, a chainsaw chain-like element, a plurality of elements threaded onto a string, a plurality of elements fastened one to the other constitute a continuum characterized by sufficient flexibility and firmness combination and variation thereof.

15. A method of performing structured sternal incision as in claim 1, wherein said surgical appliance further comprises a plurality of gripping-fixation arms, the movement of which characterized by several, controllable degrees of freedom and an actuating mechanism therefore; and wherein said positioning and leading said incising member of said surgical appliance according to a pattern is done by said actuating mechanism of said gripping-fixation arms.

16. A method of performing structured sternal incision as in claim 15, further comprising the steps of:
fastening said plurality of gripping-fixation arms to said patient;
restraining the movement of said gripping-fixation arms and setting there firm.

17. Surgical appliance as in claim 10, wherein said appliance further comprising:
a structural framework, whose movement characterized by several controllable degrees of freedom, and an actuating mechanism therefor, wherein said incising member is selected from the group consisting of: driller/blade, a beam of coherent electromagnetic radiation, a coherent jet created by stream of pressurised liquid, at least one fret-saw member, a flexible continuous band-saw blade, a chainsaw chain-like element, a plurality of elements threaded onto a string, a plurality of elements fastened one to the other that constitutes a continuum characterized by sufficient flexibility and firmness, combination and variation thereof.

18. A method of performing structured sternal incision as in claim 1, wherein said surgical appliance further comprising structural framework, whose movement characterized by several controllable degrees of freedom and a actuating mechanism therefor; and wherein said positioning and leading said incising member of said surgical appliance according to a pattern is done by conferring to said structural framework a particular three dimensional conformation by said actuating mechanism therefor.

19. A method of performing structured sternal incision as in claim 9, further comprising the steps of:
verifying the correctness of said incising member's position relatively to said pattern according to said coordinates, and
correcting said position of said incising member according to said coordinates and positioning said incising member against said pattern, if an inconsistency in said position was detected in said step of verifying.

20. A stencil for performing a structured sternal incision having a present pattern (PS).
